(19) United States
(12) Patent Application Publication
(10) Pub. No.: US 2026/0301858 A1
(43) Pub. Date: Oct. 1, 2026

(54) ARTIFICIAL INTELLIGENCE-BASED SYSTEM FOR GENERATING POCKET- CONDITIONED MOLECULES AND METHOD THEREOF

(71) Applicant: CENTELLA SCIENTIFIC PRIVATE LIMITED, HYDERABAD (IN)

(72) Inventors: RIYAZ SYED, HYDERABAD (IN); POORNA CHANDRA RAO YEDLA, HYDERABAD (IN)

(21) Appl. No.: 19/231,626

(22) Filed: Jun. 9, 2025

(30) Foreign Application Priority Data
Mar. 26, 2025 (IN) 202541028438

(51) Int. Cl. G16B 15/30; G06N 3/042; G06N 3/0475; G16B 15/20; G16B 40/20

(52) U.S. Cl. CPC G16B 15/30; G06N 3/042; G06N 3/0475; G16B 15/20; G16B 40/20

(57) ABSTRACT

The present invention discloses an artificial intelligence-based (AI-based) system for generating pocket-conditioned molecules and an artificial intelligence-based (AI-based) method thereof. The AI-based system obtains ligand data and protein pocket data for generating molecular interactions. The AI-based system aligns two-dimensional (2D) molecular representations with three-dimensional (3D) molecular conformations and two-dimensional (2D) sequences with three-dimensional (3D) coordinates to ensure a spatial consistency and a structural consistency. The AI-based system encodes the ligand data and the protein pocket data to generate topological features and spatial features. The AI-based system determines interaction between molecular structures based on incorporating positional embeddings to the encoded ligand data and protein pocket data. The AI-based system generates the pocket-conditioned molecules based on integrating the topological features and the spatial features of the encoded ligand data and protein pocket data.

### EARLIEST PRIORITY DATE

This Application claims priority from a Provisional patent application filed in India having Patent Application No. 202541028438, filed on 26 Mar. 2025 and titled “ARTIFICIAL INTELLIGENCE-BASED SYSTEM FOR GENERATING POCKET-CONDITIONED MOLECULES AND METHOD THEREOF”.

### FIELD OF INVENTION

Embodiments of the present invention relate to computational drug discovery and more particularly relate to an artificial intelligence-based (AI-based) system for generating one or more pocket-conditioned molecules and an artificial intelligence-based (AI-based) method thereof.

### BACKGROUND

Field of drug discovery relies heavily on identification of small one or more molecular structures that may effectively bind to specific protein targets. Traditional drug development methods involve extensive laboratory testing and computational docking simulations, both of which require significant time and resources. Advances in artificial intelligence (AI) and machine learning (ML) have enabled the use of one or more predictive models to accelerate this process, allowing researchers to screen vast molecular libraries for potential one or more pocket-conditioned molecules. AI-driven molecular modelling techniques have demonstrated promise in identifying the one or more pocket-conditioned molecules, optimizing molecular interactions, and reducing a time required for drug development.

However, conventional AI-based approaches in molecular discovery face limitations in accurately representing complex one or more molecular structures and molecular interactions. Many existing methods rely on two-dimensional (2D) molecular representations, which fail to capture full spatial and topological characteristics of molecular binding sites. This shortcoming may lead to inaccurate predictions of ligand-protein interactions and ineffective one or more pocket-conditioned molecules. Additionally, existing models lack adaptability to diverse molecular scaffolds, leading to suboptimal performance when applied to unique chemical spaces.

To address these challenges, the researchers have developed one or more generative machine learning models that attempt to optimize a latent space for molecular generation. Existing one or more generative machine learning models exhibit limited structural consistency, as the one or more generative machine learning models optimize the latent space for molecule generation but may not ensure spatial and structural consistency between 2D and three-dimensional (3D) molecular representations. This inconsistency may lead to inaccurate molecular interactions and unreliable predictions.

Many existing one or more generative machine learning models rely on suboptimal embedding techniques, using basic one or more positional embeddings that fail to fully capture spatial dependencies in molecular sequences, reducing prediction accuracy. Additionally, training data limitations hinder model performance, as existing one or more AI models may be trained on datasets that lack diverse molecular scaffolds, leading to poor generalization when applied to unique compounds. Moreover, the lack of reinforcement learning refinement in prior models may not iteratively improve predictions based on feedback, limiting the ability to optimize the one or more pocket-conditioned molecules effectively.

In an existing technology, a system for optimizing the latent space in the generative one or more machine learning models to improve molecular property predictions is disclosed. The system uses a computer-based approach with a latent space optimization module that trains the generative one or more machine learning models to represent various molecular properties. The system incorporates different models trained on cheminformatics data to predict synthetic accessibility, drug-likeness, ligand-protein docking poses, and bioactivity affinity. While this approach enhances the molecular generation by leveraging learned latent space representations but has several disadvantages. The system relies heavily on pre-trained models without ensuring the adaptability of generated molecules to unique chemical spaces. Additionally, the system primarily focuses on one or more static embeddings rather than incorporating advanced one or more positional embeddings, which are essential for capturing spatial dependencies in the molecular interactions.

Therefore, there is a need for a system that ensures optimal spatial and structural consistency in molecular representation, improves molecular interaction prediction accuracy, and refines one or more AI models using reinforcement learning to enhance pocket-conditioned molecule generation.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In order to overcome the above deficiencies of the prior art, the present disclosure is to solve the technical problem by providing an artificial intelligence-based (AI-based) method for generating one or more pocket-conditioned molecules.

In accordance with an embodiment of the present invention, the AI-based method for generating the one or more pocket-conditioned molecules is disclosed. In the first step, the AI-based method includes obtaining, by one or more hardware processors through a data obtaining subsystem, at least one of: ligand data comprises at least one of: two-dimensional (2D) molecular representations and three-dimensional (3D) molecular conformations and protein pocket data comprises at least one of: two-dimensional (2D) sequences and three-dimensional (3D) coordinates to generate molecular interactions. The 2D molecular representations are obtained in at least one of: simplified molecular input line entry system (SMILES), and a molecular (MOL) file format. The 2D molecular representations are configured to capture a connectivity of one or more atoms in a molecule. The 3D molecular conformations are obtained in at least one of: cartesian coordinates and crystallographic information file (CIF). The 3D molecular conformations are configured to capture a spatial arrangement of the one or more atoms. The protein pocket data is obtained in a Protein Data Bank (PDB) Format. The protein pocket data is configured to at least one of: define a linear arrangement of residues and capture a three-dimensional (3D) geometry of protein binding pockets.

In the next step, the AI-based method includes aligning, by the one or more hardware processors through a data pre-processing subsystem, at least one of: the 2D molecular representations with the 3D molecular conformations and the 2D sequences with the 3D coordinates to verify at least one of: a spatial consistency and a structural consistency.

In the next step, the AI-based method includes encoding, by the one or more hardware processors through a data encoding subsystem configured with one or more artificial intelligence (AI) models, at least one of: the ligand data and the protein pocket data to generate at least one of: one or more topological features and one or more spatial features. The one or more AI models comprise at least one of: one or more graph neural network (GNN) models, one or more transformer-based models, one or more transfer learning models, and one or more diffusion models. The one or more GNN models are configured to encode the ligand data. The one or more GNN models are selected from a group that comprises at least one of: a graph sample and aggregate (GraphSAGE) model for inductive representation learning, a graph attention network (GAT) model for enhancing spatial interaction modelling, and a graph convolutional network (GCN) for learning hierarchical one or more molecular features. The one or more transformer-based models are configured to encode the protein pocket data. The one or more transformer-based models incorporate at least one of: self-attention mechanisms and cross-attention mechanisms to capture defined-range dependencies in molecular sequences.

In the next step, the AI-based method includes determining, by the one or more hardware processors through a positional embedding subsystem, interaction between one or more molecular structures based on incorporating one or more positional embeddings to the encoded at least one of: ligand data and protein pocket data. The one or more positional embeddings are selected from a group that comprises at least one of: rotary positional embeddings (RoPE), absolute positional embeddings, and relative positional embeddings, providing at least one of: optimal structural consistency and optimal spatial consistency during a molecular interaction prediction. The positional embedding subsystem comprises rotating the encoded at least one of: ligand data and protein pocket data, in distinctive orientations to optimise generalization of the one or more AI models to predict interaction between the one or more molecular structures.

In the next step, the AI-based method includes generating, by the one or more hardware processors through a molecular generation subsystem, the one or more pocket-conditioned molecules based on integrating at least one of: the one or more topological features and the one or more spatial features of the encoded at least one of: ligand data and protein pocket data. The molecular generation subsystem comprises incorporating one or more hydrogen atoms by utilizing a base transformer attention procedure at a time of generating the one or more pocket-conditioned molecules to optimise biological relevance of the one or more pocket-conditioned molecules.

The AI-based method further includes training, by the one or more hardware processors through a model training subsystem, the one or more AI models on a universal three-dimensional (3D) molecular representation learning framework (Uni-Mol) dataset to determine at least one of: the one or more topological features and the one or more spatial features. The AI-based method further includes refining, by the one or more hardware processors through the model training subsystem, the one or more AI models utilizing reinforcement learning (RL) models trained on a structure-based drug discovery dataset to at least one of: predict ligand-protein binding affinity, optimise the generation of the one or more pocket-conditioned molecules, and optimise drug-likeness properties.

The AI-based method further includes evaluating, by the one or more hardware processors through an evaluation subsystem, the generated one or more pocket-conditioned molecules based on predefined performance metrics to determine at least one of: defined characteristics and potential information for a drug development. The predefined performance metrics comprise at least one of: binding affinity scores, a root mean square deviation (RMSD) data, and quantitative estimate of drug-likeness (QED) properties.

In accordance with an embodiment of the present invention, an artificial intelligence-based (AI-based) system for generating the one or more pocket-conditioned molecules is disclosed. The AI-based system comprises one or more servers. The one or more servers comprise the one or more hardware processors and a memory unit. The memory unit is coupled to the one or more hardware processors, wherein the memory unit comprises a plurality of subsystems in form of one or more instructions executable by the one or more hardware processors. The plurality of subsystems comprises the data obtaining subsystem, the data pre-processing subsystem, the data encoding subsystem, the positional embedding subsystem, and the molecular generation subsystem.

Yet in another embodiment, the data obtaining subsystem is configured to obtain at least one of: the ligand data comprises at least one of: the 2D molecular representations and 3D molecular conformations and the protein pocket data comprises at least one of: the 2D sequences and the 3D coordinates, for generating the molecular interactions.

Yet in another embodiment, the data pre-processing subsystem is configured to align at least one of: the 2D molecular representations with the 3D molecular conformations and the 2D sequences with the 3D coordinates to verify at least one of: the spatial consistency and the structural consistency.

Yet in another embodiment, the data encoding subsystem is configured with the one or more AI models to encode at least one of: the ligand data and the protein pocket data to generate at least one of: the one or more topological features and the one or more spatial features.

Yet in another embodiment, the positional embedding subsystem is configured to determine interaction between the one or more molecular structures based on incorporating the one or more positional embeddings to the encoded at least one of: the ligand data and the protein pocket data.

Yet in another embodiment, the molecular generation subsystem is configured to generate the one or more pocket-conditioned molecules based on integrating at least one of: the one or more topological features and the one or more spatial features of the encoded at least one of: ligand data and protein pocket data.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates an exemplary block diagram representation of a network architecture depicting an artificial intelligence-based (AI-based) system for generating one or more pocket-conditioned molecules, in accordance with an embodiment of the present disclosure;

FIG. 2 illustrates an exemplary block diagram representation of the AI-based system as shown in FIG. 1 for generating the one or more pocket-conditioned molecules, in accordance with an embodiment of the present disclosure; and

FIG. 3 illustrates an exemplary flow diagram depicting an artificial intelligence-based (AI-based) method for generating the one or more pocket-conditioned molecules, in accordance with an embodiment of the present disclosure.

Further, those skilled in the art will appreciate that elements in the figures are illustrated for simplicity and may not have necessarily been drawn to scale. Furthermore, in terms of the method steps, chemical compounds, equipment and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE PRESENT INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present invention relate to an artificial intelligence-based (AI-based) system for generating one or more pocket-conditioned molecules.

As used herein the term “pocket-conditioned molecules” refers to chemical compounds that align with essential features required for biological activity, such as, but not constricted to, at least one of: hydrogen bond donors, acceptors, hydrophobic regions, and the like. The one or more pocket-conditioned molecules are configured to fit defined target sites in drug discovery and development. The defined target sites may be, but not limited to, at least one of: enzyme active sites, receptor binding sites, ion channels, Deoxyribonucleic acid (DNA) intercalation sites, protein-protein interaction sites, and the like.

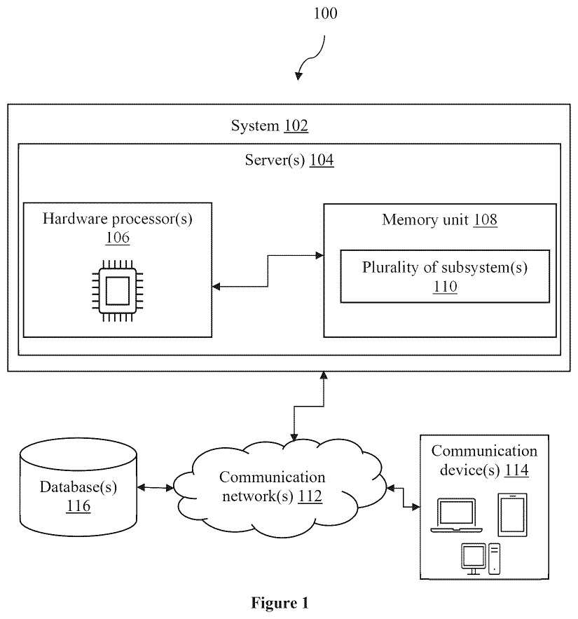

FIG. 1 illustrates an exemplary block diagram representation of a network architecture 100 depicting the AI-based system 102 for generating the one or more pocket-conditioned molecules, in accordance with an embodiment of the present disclosure.

According to an exemplary embodiment of the disclosure, the AI-based system 102 (hereinafter referred to as the system 102) for generating the one or more pocket-conditioned molecules is disclosed. The network architecture 100 may include the system 102, one or more databases 116, and one or more communication devices 114. The system 102, the one or more databases 116, and the one or more communication devices 114 may be communicatively coupled via one or more communication networks 112, ensuring seamless data transmission, processing, and decision-making. The system 102 acts as a central processing unit within the network architecture 100, responsible for generating the one or more pocket-conditioned molecules.

In an exemplary embodiment, the system 102 comprises one or more servers 104. The one or more servers 104 may comprise a combination of discrete components, an integrated circuit, an application-specific integrated circuit, a field-programmable gate array, a digital signal processor, or other suitable hardware. The one or more servers 104 comprises one or more hardware processors 106 and a memory unit 108. The memory unit 108 is operatively connected to the one or more hardware processors 106. The memory unit 108 comprises one or more instructions in the form of a plurality of subsystems 110, configured to be executed by the one or more hardware processors 106.

In an exemplary embodiment, the one or more hardware processors 106 may include, for example, microprocessors, microcomputers, microcontrollers, digital signal processors, central processing units, state machines, logic circuits, and/or any devices that manipulate data or signals based on operational instructions. Among other capabilities, the one or more hardware processors 106 may fetch and execute the one or more instructions in the memory unit 108 operationally coupled with the system 102 for performing tasks such as data processing, input/output processing, and/or any other functions. Any reference to a task in the present disclosure may refer to an operation being or that may be performed on data. The one or more hardware processors 106 are high-performance processors capable of handling large volumes of data and complex computations. The one or more hardware processors 106 may be, but not limited to, at least one of: multi-core central processing units (CPU), graphics processing units (GPUs), and the like, which enhance an ability of the system 102 to process real-time data from one or more sources simultaneously.

In an exemplary embodiment, the one or more databases 116 may configured to store and manage data related to various aspects of the system 102. The one or more databases 116 may store data associated with at least one of, but not limited to, the one or more pocket-conditioned molecules, ligand data, protein pocket data, one or more artificial intelligence (AI) models, any other information necessary for the functionality and optimization of the system 102, and the like. The one or more databases 116 may include different types of databases such as, but not limited to, relational databases (e.g., Structured Query Language (SQL) databases such as PostgresDB and Oracle® databases), non-Structured Query Language (NoSQL) databases (e.g., MongoDB, Cassandra), time-series databases (e.g., InfluxDB), an OpenSearch database, object storage systems (e.g., Amazon® S3), and the like.

In an exemplary embodiment, the one or more communication devices 114 are configured to enable one or more users to interact with the system 102. The one or more communication devices 114 may be digital devices, computing devices, and/or networks. The one or more communication devices 114 may include, but not limited to, a mobile device, a smartphone, a personal digital assistant (PDA), a tablet computer, a phablet computer, a wearable computing device, a virtual reality/augmented reality (VR/AR) device, a laptop, a desktop, and the like.

In an exemplary embodiment, the one or more communication networks 112 may be, but not limited to, a wired communication network and/or a wireless communication network, a local area network (LAN), a wide area network (WAN), a Wireless Local Area Network (WLAN), a metropolitan area network (MAN), a telephone network, such as the Public Switched Telephone Network (PSTN) or a cellular network, an intranet, the Internet, a fibre optic network, a satellite network, a cloud computing network, a combination of networks, and the like. The wired communication network may comprise, but not limited to, at least one of: Ethernet connections, Fiber Optics, Power Line Communications (PLCs), Serial Communications, Coaxial Cables, Quantum Communication, Advanced Fiber Optics, Hybrid Networks, and the like. The wireless communication network may comprise, but not limited to, at least one of: wireless fidelity (wi-fi), cellular networks (including fourth generation (4G) technologies and fifth generation (5G) technologies), Bluetooth®, ZigBee®, long-range wide area network (LoRaWAN), satellite communication, radio frequency identification (RFID), 6G (sixth generation) networks, advanced IoT protocols, mesh networks, non-terrestrial networks (NTNs), near field communication (NFC), and the like.

In an exemplary embodiment, the system 102 may be implemented by way of a single device or a combination of multiple devices that may be operatively connected or networked together. The system 102 may be implemented in hardware or a suitable combination of hardware and software.

Though few components and the plurality of subsystems 110 are disclosed in FIG. 1, there may be additional components and subsystems which is not shown, such as, but not limited to, ports, routers, repeaters, firewall devices, network devices, the one or more databases 116, network attached storage devices, assets, machinery, instruments, facility equipment, emergency management devices, image capturing devices, any other devices, and combination thereof. The person skilled in the art should not be limiting the components/subsystems shown in FIG. 1. Although FIG. 1 illustrates the system 102, and the one or more communication devices 114 connected to the one or more databases 116, one skilled in the art can envision that the system 102, and the one or more communication devices 114 may be connected to several user devices located at various locations and several databases via the one or more communication networks 112.

Those of ordinary skilled in the art will appreciate that the hardware depicted in FIG. 1 may vary for particular implementations. For example, other peripheral devices such as an optical disk drive and the like, the local area network (LAN), the wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

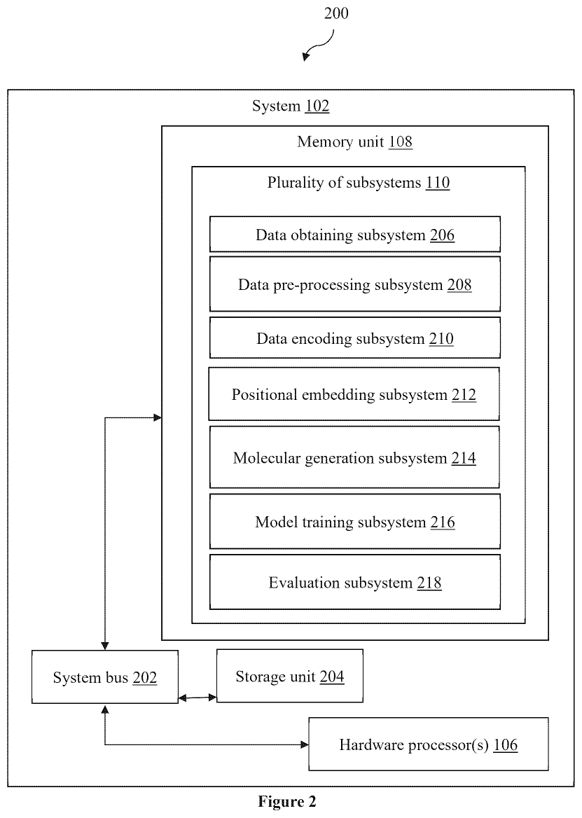

FIG. 2 illustrates an exemplary block diagram representation 200 of the system 102 as shown in FIG. 1 for generating the one or more pocket-conditioned molecules, in accordance with an embodiment of the present disclosure.

In an exemplary embodiment, the system 102 comprises the one or more servers 104, the memory unit 108, and a storage unit 204. The one or more hardware processors 106, the memory unit 108, and the storage unit 204 are communicatively coupled through a system bus 202 or any similar mechanism. The system bus 202 functions as a central conduit for data transfer and communication between the one or more hardware processors 106, the memory unit 108, and the storage unit 204. The system bus 202 facilitates the efficient exchange of information and instructions, enabling the coordinated operation of the system 102.

In an exemplary embodiment, the memory unit 108 is operatively connected to the one or more hardware processors 106. The memory unit 108 comprises the plurality of subsystems 110 in the form of the one or more instructions executable by the one or more hardware processors 106. The plurality of subsystems 110 comprises a data obtaining subsystem 206, a data pre-processing subsystem 208, a data encoding subsystem 210, a positional embedding subsystem 212, a molecular generation subsystem 214, a model training subsystem 216, and an evaluation subsystem 218. The one or more hardware processors 106 associated within the one or more servers 104, as used herein, means any type of computational circuit, such as, but not limited to, the microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 106 may also include embedded controllers, such as generic or programmable logic devices or arrays, application-specific integrated circuits, single-chip computers, and the like.

The memory unit 108 may be the non-transitory volatile memory unit and the non-volatile memory unit. The memory unit 108 may be coupled to communicate with the one or more hardware processors 106, such as being a computer-readable storage medium. The memory unit 108 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 108 includes the plurality of subsystems 110 stored in the form of the one or more instructions on any of the above-mentioned storage media and may be in communication with and executed by the one or more hardware processors 106.

The storage unit 204 may be a cloud storage or the one or more databases 116 such as those shown in FIG. 1. The storage unit 204 may store, but not limited to, recommended course of action sequences dynamically generated by the system 102. The action sequences comprise data obtaining, data pre-processing, data encoding, positional embedding, molecular generation, model training, and the like. Additionally, the storage unit 204 may retain previous action sequences for comparison and future reference, enabling continuous refinement of the system 102 over time. The storage unit 204 may be any kind of database such as, but not limited to, relational databases, dedicated databases, dynamic databases, monetised databases, scalable databases, cloud databases, distributed databases, any other databases, and a combination thereof.

In an exemplary embodiment, the data obtaining subsystem 206 is configured to obtain at least one of: ligand data and protein pocket data. The ligand data comprises a dataset of approximately 12 million molecular structures associated with one or more molecular structures. The protein pocket data comprises 3.2 million protein pockets. The ligand data may comprise, but not restricted to, at least one of: two-dimensional (2D) molecular representations, three-dimensional (3D) molecular conformations, and the like, which are essential for understanding the one or more molecular structures and molecular interactions.

The 2D molecular representations are obtained in standardized formats such as, but not limited to, at least one of: Simplified Molecular Input Line Entry System (SMILES), a Molecular (MOL) file format, and the like. Self-Referencing Embedded Strings (SELFIES) provide a robust alternative to the SMILES, ensuring that every token (atom) sequence represents a valid molecular structure associated with the one or more molecular structures. The MOL file format may provide deeper connectivity information. The 2D molecular representations are configured to encode a connectivity of one or more atoms in a molecule without explicitly defining spatial arrangement. The 2D molecular representations (Amino acid sequences) are widely employed in cheminformatics for database storage and molecular similarity searching. The 3D molecular conformations provide a spatial depiction of the one or more atoms and orientations of the one or more atoms within the molecule. The 3D molecular conformations are obtained in, but not limited to, at least one of: cartesian coordinates, crystallographic information file (CIF), and the like, which are essential for accurately modelling molecular geometries. The CIF is employed for describing crystalline structures. The 3D molecular conformations play a crucial role in computational drug construction, as the 3D molecular conformations allow for assessment of the molecular interactions, steric hindrance, and binding affinity in relation to a biological target.

The protein pocket data is acquired to enable detailed structural analysis of the defined target sites, which is crucial for understanding protein-ligand interactions. A Protein Data Bank (PDB) format is employed for storing and processing the protein pocket data, as the PDB format provides a structured representation of proteins, including atomic coordinates and connectivity information. The protein pocket data is configured to, but not limited to, at least one of: define a linear arrangement of residues, capture a three-dimensional (3D) geometry of protein binding pockets, and the like. The protein pocket data captures the linear arrangement of the residues (amino acid residues) and the 3D geometry of the protein binding pockets, which together define shape, charge distribution, and accessibility of the defined target sites where ligands may bind. The linear arrangement aids in analysing residue-specific interactions and functional motifs, while the 3D geometry is employed for docking studies and virtual screening applications. The integration of the protein pocket data with the ligand data allows the system 102 to simulate and predict the molecular interactions, which is fundamental in fields such as drug discovery, pharmacophore modelling, and structure-based drug construction.

In an exemplary embodiment, the data pre-processing subsystem 208 is configured to align, but not restricted to, at least one of: the 2D molecular representations with the 3D molecular conformations and the 2D sequences with the 3D coordinates, ensuring verification of at least one of: spatial consistency and structural consistency. This alignment is critical in molecular modelling, as the alignment validates that the 2D molecular representations (SMILES representations) accurately corresponds to the 3D molecular conformations, thereby preventing discrepancies that may affect downstream computational analyses, such as molecular docking, binding affinity predictions, and virtual screening. The verification of the spatial consistency ensures that atomic positions and the 3D molecular conformations adhere to expected geometric constraints derived from the 2D molecular representations, maintaining accurate bond angles, torsion states, and steric hindrances. The validation of the structural consistency ensures that the 2D sequences (protein sequences) match the 3D coordinates, confirming that residue placements within the defined target sites align with known crystallographic and computationally predicted positions.

In an exemplary embodiment, the data encoding subsystem 210 is configured with one or more artificial intelligence (AI) models. The data encoding subsystem 210 is configured to encode, but not restricted to, at least one of: the ligand data, the protein pocket data, and the like, ultimately generating at least one of: one or more topological features, one or more spatial features, and the like. The data encoding subsystem 210 is configured to convert raw molecular representations into structured mathematical forms that may be efficiently analysed for at least one of: drug discovery, molecular docking, and interaction modelling. The one or more AI models may comprise, but not constrained to, at least one of: one or more graph neural network (GNN) models, one or more transformer-based models, one or more transfer learning models, one or more diffusion models, and the like, to extract relevant molecular patterns from at least one of: the ligand data and the protein pocket data. The one or more GNNs are particularly effective in encoding the ligand data due to the ability to capture one or more molecular structures (molecular graphs), ensuring that atomic connectivity, bond types, and electronic interactions are maintained. The one or more GNN models are selected from a group that comprises, but not constricted to, at least one of: a graph sample and aggregate (GraphSAGE) model, a graph attention network (GAT) model, a graph convolutional network (GCN), and the like. The GraphSAGE model facilitates inductive representation learning on huge one or more molecular structures by enabling the data encoding subsystem 210 to generalize unseen one or more molecular structures. The GraphSAGE model captures the one or more topology features and the defined target sites (3D active sites) of the protein binding pockets. The GraphSAGE model is configured to generate low-dimensional vector representations for nodes (one of: atoms and residues) in the one or more molecular structures. The GAT model enhances spatial interaction modelling through adaptive attention mechanisms that prioritize critical atomic interactions. The GCN supports hierarchical extraction of one or more molecular features to distil meaningful molecular fingerprints that aid in activity prediction and pocket-conditioned molecule optimisation. The one or more molecular features may include, but not constrained to, at least one of: topological properties, electronic distribution, hydrophobicity, molecular weight, and binding affinities. The GCN captures spatial relationships in the one or more molecular structures (3D molecular graphs), modelling interactions between the one or more atoms. The GAT model and the GCN may improve the capture of spatial and chemical information in the molecule. The GAT model and the GCN may potentially provide better performance in capturing local molecular patterns and long-range dependencies in the one or more molecular structures.

The one or more transformer-based models are employed to encode the protein pocket data.

The one or more transformer-based models may incorporate, but not restricted to, at least one of: self-attention mechanisms, cross-attention mechanisms, and the like, to capture complex defined-range dependencies within molecular sequences. The one or more transformer-based models excel at modelling long-range interactions within the defined target sites (binding sites), ensuring that subtle conformational changes and allosteric effects are effectively represented. By incorporating the self-attention mechanisms, the one or more transformer-based models may weigh different amino acid residues in the molecular sequences, prioritizing the protein pocket data most relevant to ligand binding. The cross-attention mechanisms enhance protein-ligand interaction predictions by dynamically adjusting the focus between different molecular components, thereby improving the accuracy of docking simulations and structure-based drug construction. The one or more transformer-based models are configured to process sequential SMILES data, capturing long-range dependencies among atom sequences. The one or more transformer-based models process the SMILES representation and tokenizes the SMILES representation to capture connectivity and sequential relationships among the one or more atoms. Through the cross-attention mechanisms, each token (atom) selectively attends to other critical tokens, ensuring distant atoms (e.g., atom 101 relative to atom 3) exert appropriate influence during a generation process.

The one or more transfer learning models allow for the adaptation of pre-trained molecular representations to unique tasks, increasing efficiency and reducing computational costs. The one or more transfer learning models leverage knowledge gained from a substantial, general-purpose dataset associated with the one or more databases 116. The one or more transfer learning models using smaller, domain-specific models such as a Molecular Transformer (MoLFormer), may be utilized to focus learning on specific one or more molecular features and the molecular interactions. The one or more transfer learning models are trained on smaller, targeted datasets associated with the one or more databases 116, enabling the one or more transfer learning models to specialize in particular molecular properties and interactions within the context of drug discovery, thus providing better predictions for specific types of molecules and the defined target sites.

The one or more diffusion models facilitate the generation of realistic 3D molecular conformations by simulating probabilistic transformations in structural data. The one or more diffusion models may be, but not limited to, at least one of: Equivariant Diffusion Models (EDMs) and Molecular Diffusion Models (MolDiff). At least one of: the EDMs and the MolDiff are configured to learn complex molecular distributions, particularly the one or more molecular structures. The one or more diffusion models employ a noise-to-data mapping approach, where the one or more diffusion models begin by generating random noise and iteratively refine the random noise towards a realistic molecular distribution, thus learning the distribution of the one or more molecular structures in 3D space.

The one or more transformer-based models focus on sequence-based data (SMILES), but future enhancements may explore vision-based architectures such as graph neural networks with convolutional layers for directly processing the one or more molecular structures, which may more naturally integrate both 2D molecular information and 3D molecular information.

In an exemplary embodiment, the positional embedding subsystem 212 is configured to determine interaction between the one or more molecular structures. The positional embedding subsystem 212 is configured to incorporate one or more positional embeddings into the encoded at least one of: ligand data and protein pocket data to enhance the precision of a molecular interaction prediction. The one or more positional embeddings are configured to ensure that the one or more molecular structures maintain at least one of: optimal structural consistency and optimal spatial consistency during the molecular interaction prediction. The one or more positional embeddings are selected from a group that comprises, but not limited to, at least one of: rotary positional embeddings (RoPE), absolute positional embeddings, relative positional embeddings, and the like.

The RoPE is particularly effective in preserving rotational invariance, making the RoPE well-suited for modelling the molecular interactions that are orientation-dependent. The RoPE embeddings add spatial awareness to the one or more AI models, enabling the one or more AI models to capture positional relationships between the one or more atoms in at least one of: the one or more molecular structures and the residues in the protein pockets. This is particularly important for accurately modelling the molecular interactions. The absolute positional embeddings encode fixed positions of the one or more atoms and the residues within the one or more molecular structures. The relative positional embeddings capture spatial relationships between the one or more atoms and the one or more residues dynamically, improving the ability of the one or more AI models to infer long-range molecular dependencies. One or more transformer-XL models employ the relative positional embeddings, providing another way to capture long-distance dependencies. The one or more positional embeddings allow the system 102 to maintain geometric coherence when predicting how the ligands interact with the defined target sites, thereby optimizing drug discovery workflows such as docking simulations, virtual screening, and ligand-based molecular construction.

The positional embedding subsystem 212 comprises rotating the encoded at least one of: ligand data and protein pocket data in distinctive orientations, ensuring that the 2D molecular representations remain robust to spatial transformations. By systematically adjusting molecular orientations during training, the one or more AI models improve predictive performance across a diverse range of the one or more molecular structures, including conformationally flexible ligands and dynamic protein pockets. This rotational approach ensures that the one or more AI models may not become biased toward specific molecular conformations but instead learn generalizable interaction patterns that apply across different one or more pocket-conditioned molecules and target protein pockets. This alignment facilitates efficient cross-attention mechanism utilization within the one or more transformer-based models, minimizing an attention matrix size and computational requirements. This approach guarantees accurate spatial correspondence and consistency between the one or more molecular structures and the 2D molecular representations.

In an exemplary embodiment, the molecule generation subsystem 214 is configured to generate the one or more pocket-conditioned molecules. The molecule generation subsystem 214 integrates at least one of: the one or more topological features and the one or more spatial features extracted from the encoded at least one of: ligand data and protein pocket data, ensuring that the generated one or more pocket-conditioned molecules align with desired pharmacophore characteristics. The molecule generation subsystem 214 generates the one or more pocket-conditioned molecules that maintain at least one of: optimal binding affinity, geometric compatibility, and functional group alignment with the target protein pockets.

The molecular generation subsystem 214 incorporates one or more hydrogen atoms by using a base transformer attention procedure. The molecular generation subsystem 214 refines stereochemistry, polarity, and electronic distribution of the one or more pocket-conditioned molecules, ensuring that the one or more pocket-conditioned molecules exhibit biologically relevant interactions with the biological target. The transformer-based attention procedure dynamically assesses and adjusts the placement of the one or more hydrogen atoms in real time, optimizing factors such as, but not limited to, at least one of: hydrogen bonding potential, molecular stability, and overall drug-like properties. The drug-like properties refer to physicochemical and pharmacokinetic characteristics that determine suitability of the one or more pocket-conditioned molecules as one or more drug candidates. The one or more hydrogen atoms significantly influence hydrophobicity and steric interactions within binding protein pockets. Explicitly modelling the one or more hydrogen atoms during the generation process leads to more accurate and biologically relevant 3D molecular conformations, providing improved results over post-processing methods. By fine-tuning the one or more molecular features during the generation process, the molecular generation subsystem 214 enhances the pharmacological viability of the one or more pocket-conditioned molecules, making the one or more pocket-conditioned molecules suitable for lead optimization, drug discovery, and computational medicinal chemistry applications.

The one or more hydrogen atoms are explicitly included during molecule generation, this may be further explored by testing different methods for handling hydrogen in molecular modelling. For instance, different representations and treatment of hydrogen bonding and impact of the one or more hydrogen atoms on molecular stability may provide insights into optimizing the 3D molecular conformations. Post-generation hydrogen handling, where the one or more hydrogen atoms are added after optimising a core molecular structure associated with the one more molecular structures, may be evaluated in combination with a current in-generation approach to assess potential improvements in the overall binding affinity and molecular stability.

In an exemplary embodiment, the model training subsystem 216 is configured to train the one or more AI models on a universal three-dimensional (3D) molecular representation learning framework (Uni-Mol) dataset. The model training subsystem 216 is configured to train the one or more AI models to extract and determine at least one of: the one or more topological features and the one or more spatial features essential for accurately representing the one or more molecular structures and the molecular interactions. The Uni-Mol dataset enables the one or more AI models to learn generalizable molecular embeddings, ensuring robust feature extraction across at least one of: the diverse ligand data and protein pocket data. The one or more AI models are trained to develop a foundational understanding of the one or more molecular structures and the molecular interactions, which may then be fine-tuned for specific tasks, reducing the overall training time and improving performance.

The one or more AI models are trained on substantial datasets such as the Uni-Mol dataset, expanding the datasets and incorporating data from other sources (e.g., protein-ligand complex databases associated with the one or more databases 116, clinical trial data, and natural product libraries) may generalize the one or more AI models further. This may also uncover the one or more AI models to a broader range of binding scenarios, potentially increasing predictive power.

Integrating data from different domains, such as transcriptomic data and proteomic data may allow the one or more AI models to better understand the biological context of protein-ligand interactions. This may be especially valuable for targeting the protein pockets involved in diseases with complex molecular mechanisms, such as cancer and neurodegenerative diseases. Multi-task learning approaches, where the one or more AI models simultaneously learns to predict at least one of: binding affinity, bioactivity, and toxicity, may be employed. This may allow for more comprehensive drug construction workflows where multiple outcomes are optimized together.

In an exemplary embodiment, the model training subsystem 216 is configured to refine the one or more AI models through reinforcement learning (RL) models trained on a structure-based drug discovery dataset (i.e., CrossDocked 2020 dataset). The structure-based drug discovery dataset may comprise, but not limited to, at least one of: molecular structures, protein-ligand complexes, and bioactivity data employed to train the one or more AI models for predicting drug interactions. The RL models fine-tune the one or more AI models to, but not limited to, at least one of: predict ligand-protein binding affinity, optimise the generation of the one or more pocket-conditioned molecules, optimise drug-likeness properties, and the like. The ligand-protein binding affinity assesses how strongly the ligands bind to the target protein pockets, thereby improving virtual screening and molecule optimization processes. The RL models optimize the generation of the one or more pocket-conditioned molecules, ensuring that the one or more pocket-conditioned molecules maintain high specificity, stability, and functional relevance to the respective the target protein pockets. The RL models optimise the drug-likeness properties, adjusting the one or more molecular structures to enhance absorption, distribution, metabolism, excretion, and toxicity (ADMET) profiles, synthetic feasibility, and overall bioavailability.

In an exemplary embodiment, the evaluation subsystem 218 is configured to evaluate the generated one or more pocket-conditioned molecules. The evaluation subsystem 218 evaluates the generated one or more pocket-conditioned molecules based on predefined performance metrics, which are critical in determining defined characteristics and potential of the generated one or more pocket-conditioned molecules for further development in the drug discovery. The predefined performance metrics may comprise, but not limited to, at least one of: binding affinity scores, a root mean square deviation (RMSD) data, quantitative estimate of drug-likeness (QED) properties, and the like. The binding affinity scores indicate how effectively the one or more molecular structures binds to the target protein pockets, providing valuable insights into the potential efficacy of the one or more pocket-conditioned molecules as one or more drug candidates. Additionally, the RMSD data is configured to measure the structural similarity between the generated one or more pocket-conditioned molecules and the protein binding pockets, allowing for an assessment of the precision and alignment of the one or more pocket-conditioned molecules with the defined target sites. The RMSD data assesses the deviation between predicted one or more molecular structures and actual one or more molecular structures. The QED properties evaluate the overall desirability of the one or more pocket-conditioned molecules based on properties such as molecular size, lipophilicity, and the presence of functional groups that contribute to bioavailability and safety.

The one or more AI models may be further optimized for specific protein families, such as G-protein coupled receptors (GPCRs), kinases, and ion channels, which are important drug targets. Tailoring the one or more AI models to better understand the structural peculiarities of the protein families may prepare the one or more AI models more effective for constructing targeted drugs. For proteins involved in allosteric modulation and multi-site binding, enhancing the one or more AI models to predict interactions at the defined target sites and account for the allosteric effects may unlock new avenues for the drug discovery.

One or more active learning techniques may be employed to refine the one or more AI models in real-time as new data becomes available. For instance, as new experimental data on protein-ligand binding is generated, the one or more AI models may be adapted and fine-tuned to account for the latest discoveries, ensuring that the predictions remain accurate and relevant. Implementing the RL models may optimize the drug construction process by using a reward-based system to optimize molecular properties. This approach improves efficiency and accuracy in generating the one or more pocket-conditioned molecules. The one or more AI models may receive feedback based on the accomplishment of the generated one or more pocket-conditioned molecules in experimental assays (e.g., binding affinity and toxicity tests), and a network may improve the predictions iteratively.

By leveraging at least one of: cloud computing, high-performance one or more GPUs, distributed learning techniques, and the like, may ensure that the one or more AI models may handle large-scale data processing and molecular generation tasks efficiently. The employment of model compression and quantization techniques may also improve the efficiency of the one or more AI models, making the one or more AI models feasible to deploy in environments with limited computational resources.

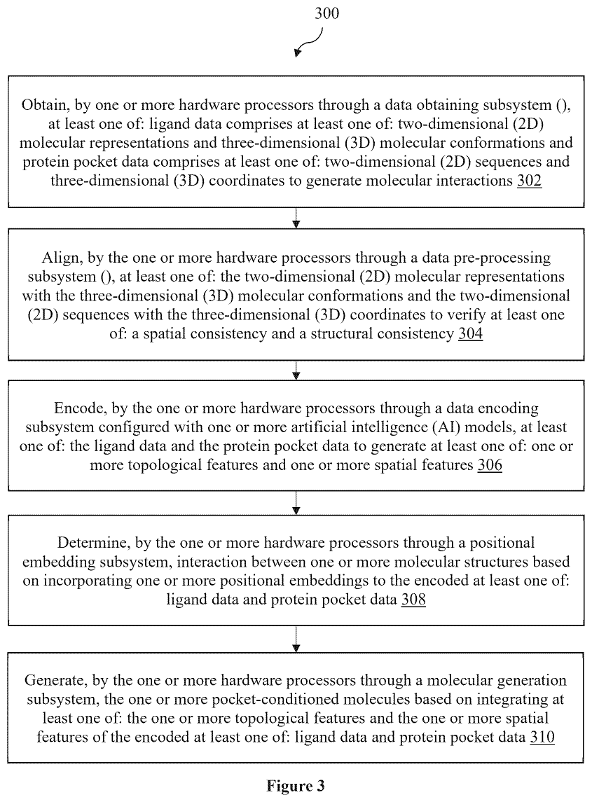

FIG. 3 illustrates an exemplary flow diagram depicting an artificial intelligence-based (AI-based) method 300 for generating the one or more pocket-conditioned molecules, in accordance with an embodiment of the present disclosure.

According to an exemplary embodiment of the disclosure, the AI-based method 300 for generating the one or more pocket-conditioned molecules is disclosed. At step 302, the AI-based method 300 includes obtaining, by the one or more hardware processors through the data obtaining subsystem, at least one of: the ligand data and the protein pocket data. The ligand data comprises at least one of: the 2D molecular representations (showing how the one or more atoms are connected) and the 3D molecular conformations (capturing the spatial arrangement). The protein pocket data comprises at least one of: the 2D sequences of amino acids and the 3D coordinates to generate the molecular interactions. At least one of: the ligand data and the protein pocket data enable the system to understand how the one or more molecular structures interact at a detailed level.

At step 304, the AI-based method 300 includes aligning at least one of: the 2D molecular representations with the 3D molecular conformations and the 2D sequences with the 3D coordinates using the one or more hardware processors through the data pre-processing subsystem. By verifying at least one of: the spatial consistency and the structural consistency, the data pre-processing subsystem ensures that molecular data is correctly formatted and reliable for further processing and analysis.

At step 306, the AI-based method 300 involves encoding at least one of: the ligand data and the protein pocket data using the one or more hardware processors through the data encoding subsystem configured with the one or more AI models. The data encoding subsystem processes at least of: the ligand data and the protein pocket data to generate at least one of: the one or more topological features and the one or more spatial features. At least one of: the one or more topological features and the one or more spatial features assist in understanding the molecular interactions, aiding in drug discovery and biomolecular analysis by capturing structural relationships and interaction potential.

At step 308, the AI-based method 300 includes determining the molecular interactions using the one or more hardware processors through the positional embedding subsystem. The positional embedding subsystem enhances the encoded at least one of: ligand data and protein pocket data by incorporating the one or more positional embeddings. The one or more positional embeddings are selected from a group that comprises, but not limited to, at least one of: the RoPE, the absolute positional embeddings, the relative positional embeddings, and the like. The one or more positional embeddings maintain at least one of: the optimal structural consistency and the optimal spatial consistency, ensuring the accurate molecular interaction predictions.

At step 310, the AI-based method 300 utilizes the one or more hardware processors through the molecular generation subsystem to generate the one or more pocket-conditioned molecules. The molecular generation subsystem integrates key molecular characteristics, including at least one of: the one or more topological features and the one or more spatial features derived from the encoded at least one of: ligand data and protein pocket data. This ensures that the one or more pocket-conditioned molecules align with essential pharmacophore properties, optimizing potential effectiveness in the drug discovery and molecular interaction studies.

The AI-based method 300 further includes training the one or more AI models using the one or more hardware processors through the model training subsystem. This training process utilizes the Uni-Mol dataset to extract and determine at least one of: the one or more topological features and the one or more spatial features.

The AI-based method 300 further includes refining the one or more AI models using the one or more hardware processors through the model training subsystem. This refinement process leverages the RL models that are trained on the structure-based drug discovery dataset. The model training subsystem is configured to enhance the capabilities of the one or more AI models in at least one of: predicting the ligand-protein binding affinity, optimizing the generation of the one or more pocket-conditioned molecules, and improving the drug-likeness properties.

The AI-based method 300 further includes evaluating the generated one or more pocket-conditioned molecules using the one or more hardware processors through the evaluation subsystem. This evaluation is conducted based on predefined performance metrics to assess critical aspects of molecular construction, ensuring suitability for drug development. The predefined performance metrics may comprise, but not restricted to, at least one of: the binding affinity scores, the RMSD data, and quantitative estimate of the QED properties. This step ensures that the generated one or more pocket-conditioned molecules align with pharmacological and structural requirements, enhancing viability for further research and application.

Numerous advantages of the present disclosure may be apparent from the discussion above. In accordance with the present disclosure, the system for generating the one or more pocket-conditioned molecules is disclosed. The system generates one or more molecular graphs (topology) and the 3D molecular conformations together, eliminating the need for external graph reconstruction and reducing potential instabilities. The system utilizes large-scale pretraining on millions of 3D molecular conformations and the protein pockets, enabling robust learning of chemical properties and spatial properties, enhancing downstream task performance, and supporting fine-tuning across specialized datasets. The system optimises molecular generation, reducing false positives and increasing the likelihood of synthesizing viable one or more pocket-conditioned molecules. The system also streamlines the drug development process by automating molecule construction, reducing reliance on trial-and-error synthesis, and accelerating lead optimization.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I/We claim:

1. An artificial intelligence-based (AI-based) method for generating one or more pocket-conditioned molecules, comprising:
obtaining, by one or more hardware processors through a data obtaining subsystem, at least one of: ligand data comprises at least one of: two-dimensional (2D) molecular representations and three-dimensional (3D) molecular conformations and protein pocket data comprises at least one of: two-dimensional (2D) sequences and three-dimensional (3D) coordinates to generate molecular interactions;
aligning, by the one or more hardware processors through a data pre-processing subsystem, at least one of: the two-dimensional (2D) molecular representations with the three-dimensional (3D) molecular conformations and the two-dimensional (2D) sequences with the three-dimensional (3D) coordinates to verify at least one of: a spatial consistency and a structural consistency;
encoding, by the one or more hardware processors through a data encoding subsystem configured with one or more artificial intelligence (AI) models, at least one of: the ligand data and the protein pocket data to generate at least one of: one or more topological features and one or more spatial features;
determining, by the one or more hardware processors through a positional embedding subsystem, interaction between one or more molecular structures based on incorporating one or more positional embeddings to the encoded at least one of: ligand data and protein pocket data; and
generating, by the one or more hardware processors through a molecular generation subsystem, the one or more pocket-conditioned molecules based on integrating at least one of: the one or more topological features and the one or more spatial features of the encoded at least one of: ligand data and protein pocket data.

2. The artificial intelligence-based (AI-based) method as claimed in claim 1, comprising:
training, by the one or more hardware processors through a model training subsystem, the one or more artificial intelligence (AI) models on a universal three-dimensional (3D) molecular representation learning framework (Uni-Mol) dataset to determine at least one of: the one or more topological features and the one or more spatial features; and
refining, by the one or more hardware processors through the model training subsystem, the one or more artificial intelligence (AI) models utilizing reinforcement learning (RL) models trained on a structure-based drug discovery dataset to at least one of: predict ligand-protein binding affinity, optimise the generation of the one or more pocket-conditioned molecules, and optimise drug-likeness properties.

3. The artificial intelligence-based (AI-based) method as claimed in claim 1, comprising:
evaluating, by the one or more hardware processors through an evaluation subsystem, the generated one or more pocket-conditioned molecules based on predefined performance metrics to determine at least one of: defined characteristics and potential information for a drug development,
the predefined performance metrics comprise at least one of: binding affinity scores, a root mean square deviation (RMSD) data, and quantitative estimate of drug-likeness (QED) properties.

4. The artificial intelligence-based (AI-based) method as claimed in claim 1, wherein
the two-dimensional (2D) molecular representations are obtained in at least one of: simplified molecular input line entry system (SMILES), and a molecular (MOL) file format, configured to capture a connectivity of one or more atoms in a molecule;
the three-dimensional (3D) molecular conformations are obtained in at least one of: cartesian coordinates and crystallographic information file (CIF), configured to capture a spatial arrangement of the one or more atoms; and
the protein pocket data is obtained in a Protein Data Bank (PDB) Format, configured to at least one of: define the linear arrangement of residues and capture a three-dimensional (3D) geometry of protein binding pockets.

5. The artificial intelligence-based (AI-based) method as claimed in claim 1, wherein the one or more artificial intelligence (AI) models comprise at least one of:
one or more graph neural network (GNN) models configured to encode the ligand data;
the one or more graph neural network (GNN) models selected from a group comprises at least one of:
a graph sample and aggregate (GraphSAGE) model for inductive representation learning;
a graph attention network (GAT) model for enhancing spatial interaction modelling; and
a graph convolutional network (GCN) for learning hierarchical one or more molecular features;
one or more transformer-based models configured to encode the protein pocket data,
the one or more transformer-based models incorporate at least one of: self-attention mechanisms and cross-attention mechanisms to capture defined-range dependencies in molecular sequences;
one or more transfer learning models; and
one or more diffusion models.

6. The artificial intelligence-based (AI-based) method as claimed in claim 1, wherein the one or more positional embeddings are selected from a group comprises at least one of: rotary positional embeddings (RoPE), absolute positional embeddings, and relative positional embeddings, providing at least one of: optimal structural consistency and optimal spatial consistency during a molecular interaction prediction.

7. The artificial intelligence-based (AI-based) method as claimed in claim 1, wherein the positional embedding subsystem comprises:
Rotating the encoded at least one of: ligand data and protein pocket data, in distinctive orientations to optimise generalization of the one or more artificial intelligence (AI) models to predict interaction between the one or more molecular structures.

8. The artificial intelligence-based (AI-based) method as claimed in claim 1, wherein the molecular generation subsystem comprises:
incorporating one or more hydrogen atoms by utilizing a base transformer attention procedure at a time of generating the one or more pocket-conditioned molecules to optimise biological relevance of the one or more pocket-conditioned molecules.

9. An artificial intelligence-based (AI-based) system for generating one or more pocket-conditioned molecules, comprising:
one or more servers, comprising:
one or more hardware processors; and
a memory unit coupled to the one or more hardware processors, wherein the memory unit comprises a plurality of subsystems in form of one or more instructions executable by the one or more hardware processors, and wherein the plurality of subsystems comprises:
a data obtaining subsystem configured to obtain at least one of: ligand data comprises at least one of: two-dimensional (2D) molecular representations and three-dimensional (3D) molecular conformations and protein pocket data comprises at least one of: two-dimensional (2D) sequences and three-dimensional (3D) coordinates, for generating molecular interactions;
a data pre-processing subsystem configured to align at least one of: the two-dimensional (2D) molecular representations with the three-dimensional (3D) molecular conformations and the two-dimensional (2D) sequences with the three-dimensional (3D) coordinates to verify at least one of: a spatial consistency and a structural consistency;
a data encoding subsystem configured with one or more artificial intelligence (AI) models to encode at least one of: the ligand data and the protein pocket data to generate at least one of: one or more topological features and one or more spatial features;
a positional embedding subsystem configured to determine interaction between one or more molecular structures based on incorporating one or more positional embeddings to the encoded at least one of: ligand data and protein pocket data; and
a molecular generation subsystem configured to generate the one or more pocket-conditioned molecules based on integrating at least one of: the one or more topological features and the one or more spatial features of the encoded at least one of: ligand data and protein pocket data.

10. The artificial intelligence-based (AI-based) system as claimed in claim 9, comprising:
a model training subsystem configured to:
train the one or more artificial intelligence (AI) models on a universal three-dimensional (3d) molecular representation learning framework (Uni-Mol) dataset to determine at least one of: the one or more topological features and the one or more spatial features; and
refine the one or more artificial intelligence (AI) models utilizing reinforcement learning (RL) models trained on a structure-based drug discovery dataset to at least one of: predict ligand-protein binding affinity, optimise the generation of the one or more pocket-conditioned molecules, and optimise drug-likeness properties.

11. The artificial intelligence-based (AI-based) system as claimed in claim 9, comprising:
an evaluation subsystem configured to evaluate the generated one or more pocket-conditioned molecules based on predefined performance metrics to determine at least one of: defined characteristics and potential information for a drug development,
the predefined performance metrics comprise at least one of: binding affinity scores, a root mean square deviation (RMSD) data, and quantitative estimate of drug-likeness (QED) properties.

12. The artificial intelligence-based (AI-based) system as claimed in claim 9, wherein the one or more artificial intelligence (AI) models comprise at least one of:
one or more graph neural network (GNN) models configured to encode the ligand data;
the one or more graph neural network (GNN) models selected from a group comprises at least one of:
a graph sample and aggregate (GraphSAGE) model for inductive representation learning;
a graph attention network (GAT) model for enhancing spatial interaction modelling; and
a graph convolutional network (GCN) for learning hierarchical one or more molecular features;
one or more transformer-based models configured to encode the protein pocket data,
the one or more transformer-based models incorporate at least one of: self-attention mechanisms and cross-attention mechanisms to capture defined-range dependencies in molecular sequences;
one or more transfer learning models; and
one or more diffusion models.
